(19) United States
(12) Patent Application Publication
(10) Pub. No.: US 2011/0196205 A1
(43) Pub. Date: Aug. 11, 2011

(54) SURGICAL PORTAL LOCKING SYSTEM

(75) Inventors: Peter Hathaway, Lebanon, CT (US); Eric Taylor, East Hampton, CT (US)

(73) Assignee: Tyco Healthcare Group LP

(21) Appl. No.: 12/987,327

(22) Filed: Jan. 10, 2011

Related U.S. Application Data
(60) Provisional application No. 61/301,775, filed on Feb. 5, 2010.

(51) Int. Cl. A61B 1/32

(52) U.S. Cl. 600/201

(57) ABSTRACT

A surgical portal apparatus includes a portal having an elongated portal member and defining a portal passage for reception of a surgical object, and a locking base. The locking base includes a flange segment defining a longitudinal axis and having a longitudinal opening for permitting reciprocal longitudinal passage of the portal member and a locking segment mounted to the flange segment. The flange segment is dimensioned and configured for positioning against body tissue. The locking segment is dimensioned to selectively engage the portal member and establish a secured relation therewith to selectively secure the portal at a predetermined longitudinal position with respect to the longitudinal axis of the locking base. The flange segment of the locking base may include an adhesive coating for adhesive engagement with the body tissue.

### CROSS REFERENCE TO RELATED APPLICATION

The present application claims the benefit of and priority to U.S. Provisional Application Ser. No. 61/301,775 filed on Feb. 5, 2010, the entire contents of which are incorporated herein by reference.

### BACKGROUND

1. Technical Field

The present disclosure relates to a surgical apparatus. More particularly, the present disclosure relates to a surgical portal apparatus for permitting introduction of a portal into a body wall and maintaining the portal in fixed relation during a surgical procedure.

2. Background of Related Art

In minimally invasive surgical procedures, including endoscopic and laparoscopic surgeries, a surgical portal permits the introduction of a variety of surgical instruments into a body cavity or opening. A surgical portal is introduced through a cavity or incision to provide access to an underlying surgical site in the body. The incision is typically made using an obturator having a blunt or sharp radius within the passageway of the surgical portal. For example, a trocar has a cannula, a tube of rigid, thin wall construction, through which an obturator may be distally passed. The obturator is utilized to penetrate a body wall, such as an abdominal wall, or to introduce the portal through the body wall, and is then removed to permit introduction of surgical instrumentation through the portal to perform the surgical procedure.

These procedures may present issues with respect to maintenance of the portal within the body wall, particularly, when exposed to a pressurized environment and/or over insertion of the portal and possible impingement of underlying body organs.

### SUMMARY

Accordingly, the present disclosure is directed to a surgical portal apparatus including a portal having an elongated portal member and defining a portal passage for reception of a surgical object, and a locking base. The locking base includes a flange segment defining a longitudinal axis and has a longitudinal opening for permitting reciprocal longitudinal passage of the portal member, and a locking segment mounted to the flange segment. The flange segment is dimensioned and configured for positioning against body tissue. The locking segment is dimensioned to selectively engage the portal member and establish a secured relation therewith to selectively secure the portal at a predetermined longitudinal position with respect to the longitudinal axis of the locking base. The flange segment of the locking base may include an adhesive coating for adhesive engagement with the body tissue.

The portal member may include a fixation element. The fixation element may be dimensioned to engage the body tissue. The fixation element may include an expandable element. The expandable element may be a balloon member. Alternatively, the expandable element may be an expandable mesh.

A manual manipulative member may be mounted adjacent the locking segment. The manipulative member may be movable to bias the locking segment in at least a radial inward direction relative to the longitudinal axis to engage the portal member. The locking segment may include a locking collet. The locking collet defines a collet passage for reception of the portal member. The locking collet is adapted to move between an initial condition permitting movement of the portal member within the collet passage and a clamped condition securing the portal member relative to the collet passage. The locking segment may include collet segments. The collet segments may be adapted for radial inward movement relative to the longitudinal axis during movement of the manipulative member. The manipulative member may include a threaded member, which cooperatively engages corresponding threads of the locking collet.

In another embodiment, a surgical portal apparatus includes a portal member defining proximal and distal ends and having a portal passage for reception of a surgical object, and a locking base. The portal member has an expandable fixation element adjacent the distal end, which is expandable to engage body tissue. The locking base includes a flange segment defining a longitudinal opening for permitting reciprocal longitudinal movement of the portal member, and a locking collet. The flange segment may be dimensioned to engage body tissue opposing the body tissue engaged by the portal member. The locking collet may define a collet passage for reception of the portal member. The locking collet may be adapted to selectively transition between an initial condition permitting passage of the portal member through the collet passage and a clamped condition where the locking collet secures the portal member within the locking base. A manual manipulative member may be mounted to the locking collet. The manipulative member is selectively movable to selectively cause transition of the locking collet between the initial condition ad the clamped condition. The locking collet may be normally biased toward the initial condition. The flange segment may include apertures therethrough to permit visualization through the flange segment. The locking base further may include an insufflation port.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a front elevational view of the surgical portal system in accordance with the present disclosure illustrating the portal and the locking base;

FIG. 2 is a top perspective view of the locking base of the surgical portal system of FIG. 1;

FIG. 3 is an enlarged isolated view of the area of detail identified in FIG. 2;

FIG. 4 is a front elevational view of the surgical portal system illustrating the locking segment of the locking base in a secured condition and the fixation element of the portal in an expanded condition; and

FIG. 5 is a front elevational view of another embodiment of a surgical portal system in accordance with the present disclosure.

### DETAILED DESCRIPTION OF EMBODIMENTS

The portal apparatus of the present disclosure contemplates the introduction of various types of instrumentation adapted for insertion through a trocar and/or cannula assembly. Examples of instrumentation include, but are not limited to, clip appliers, graspers, dissectors, retractors, staplers, laser probes, photographic devices, endoscopes and laparoscopes, tubes, and the like. Such instruments will collectively be referred to as “instruments”, “instrumentation” or “surgical objects” which also may include the hand of a clinician.

The portal apparatus may be any suitable cannula assembly used in laparoscopic or arthroscopic procedures. The portal apparatus may also be adapted to receive the hand of a surgeon during, e.g., a minimally invasive laparoscopic hand assisted procedure. The portal apparatus includes a locking or clamping mechanism to secure a portal member of the apparatus relative to the body tissue and also permit selected advancement or retracting movement of the portal member relative to the underlying target tissue site.

In the following description, as is traditional, the term “proximal” or “trailing” refers to the portion of the device closer to the operator while the term “distal” or “leading” refers to the portion of the device further from the operator.

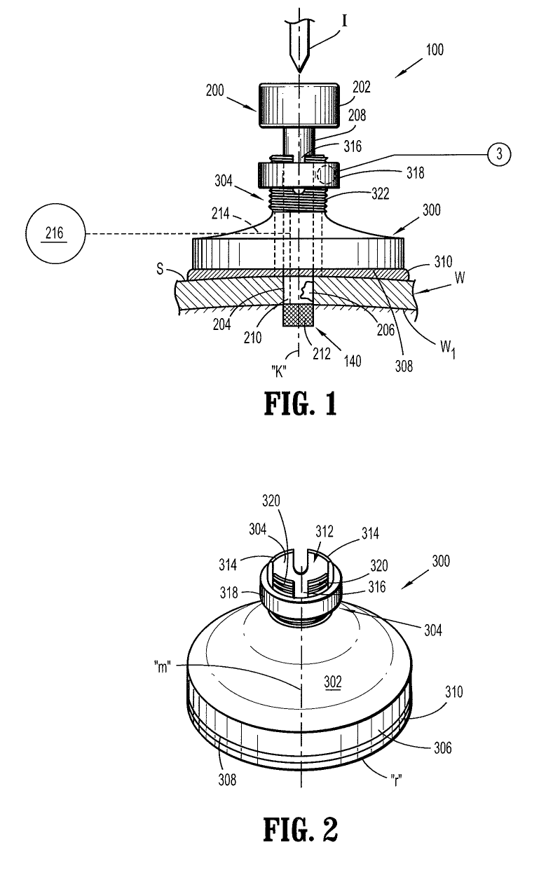

Referring now to the drawings, in which like reference numerals identify identical or substantially similar parts throughout the several views, FIG. 1 illustrates an exemplary embodiment of the portal apparatus 100 in accordance with the principles of the present disclosure. Portal apparatus 100 may include a laparoscopic cannula assembly utilized in conjunction with a laparoscopic surgical procedure where the peritoneal cavity is insufflated with a suitable gas, e.g., CO₂, to raise the cavity wall from the internal organs therein. The cannula assembly may be used with an obturator assembly (not shown) which is a sharp pointed instrument positionable within the passageway of the cannula assembly. The obturator assembly is utilized to penetrate the abdominal wall and then subsequently removed from the cannula assembly to permit introduction of the surgical instrumentation utilized to perform the procedure. In the alternative, portal apparatus 100 may be an arthroscopic cannula assembly used in connection with an arthroscopic surgical procedure.

Portal apparatus 100 includes portal 200 and locking base 300 which receives the portal 200 and selectively secures the portal 200 at a predetermined longitudinal position. Portal 200 includes portal housing 202 and elongated portal member 204 extending from the portal housing 202. Portal housing 202 may include multiple housing segments connected to each other via convention means or may be a single component integrally or monolithically formed. Portal housing 202 and portal member 204 define longitudinal passage 206 (shown in partial cut-away of FIG. 1) extending along longitudinal axis “k” defined by the portal 200.

Portal member 204 may be a sleeve member having proximal or trailing end 208 and distal or leading end 210 of the portal member 204. Portal member 204 may be a separate component connected to portal housing 202 or may be monolithically formed with the portal housing 202. Portal member 204 and portal housing 202 may be releasably connected through a variety of mechanisms including, e.g., through a bayonet lock, threaded connection, or the like.

Portal member 204 may be formed of stainless steel or other rigid materials such as a polymeric material or the like. Portal member 204 may be clear or opaque. The diameter of portal member 204 may vary, but typically ranges from about 3 to about 15 mm when used in a laparoscopic or arthroscopic technique. If used in a hand assisted minimally invasive approach, the diameter of portal member may be substantially greater than 15 mm.

Portal apparatus 100 may include one or more seals or valves, e.g., at least partially mounted within portal housing 202. One seal may be an instrument seal defining a seal passage to receive the surgical object and form a substantial seal about the object. Another seal or valve is a zero closure valve that is adapted to close longitudinal passage 206 of portal 200 in the absence of the surgical object. Suitable instrument seals and zero closure valves are disclosed in commonly assigned U.S. Pat. No. 6,702,787 to Racenet, the entire contents of which disclosure is hereby incorporated by reference herein.

Portal 200 may include fixation element 212 disposed at the distal end 210 of portal member 204. Fixation element 212 may be configured to selectively expand and contract to assist in retention of portal 200 relative to body wall W. In one embodiment, fixation element 212 is a deployable mesh adapted for selective expansion through movement of a deployment member (not shown). In another embodiment, fixation element 212 may comprise a shape memory material adapted to expand in response to achieving a temperature threshold such as being exposed to body temperature when introduced within the body. Suitable shape memory materials include pseudo elastic metals such as nickel-titanium (NiTi) alloys. In another embodiment, fixation element 212 is an expandable balloon adapted for expansion through introduction of liquids into the expandable balloon through conduit 214 (shown in schematics) supplied by fluid source 216. Fixation element 212 when expanded is adapted to contact the internal side W₁ of body wall W to assist in retention of portal member 202 relative to the body wall W and minimize the potential of retropulsion of the portal member 202 from the body wall W. As a further alternative, fixation element 212 may be a compressible foam or gel. Suitable fixation elements 212 which may be incorporated within portal 200 are disclosed in commonly assigned U.S. Pat. No. 5,330,497 to Freitas, the entire contents of which disclosure are hereby incorporated by reference herein.

With reference to FIGS. 1-2, locking base 300 is configured to releasably secure portal 200 at a predetermined longitudinal position. In particular, locking base 300 is configured to maintain portal 200 stationary and at a predetermined depth of axial insertion within the body wall W of the patient. Locking base 300 includes flange segment 302 and locking segment 304 depending outwardly from the flange segment 302. Flange segment 302 defines central longitudinal axis “m” extending through locking segment 304, and has end wall 306. End wall 306 has contacting surface 308 configured to contact body surface “S” of a body wall “W”. Flange segment 302 of locking base 300 may be substantially conical in shape or may be other shapes which provide an increased surface area to the end wall 306. End wall 306 may also include an adhesive coating or layer 310 disposed on contact surface 308. Suitable adhesives include cyanoacrylate or a polyethylene glycol polymer which may be disposed on a portion of, or all, of end wall 306. Adhesive coating 310 may be a pressure sensitive adhesive, such as an adhesive tape affixed or bonded to end wall 306. Adhesive coating 310 may function to temporarily secure locking base 300 to body surface “S” during application of the locking base 300 and manipulation and positioning of portal apparatus 200. A protective liner, e.g., a release liner or backing sheet “r” (FIG. 2), may be disposed on adhesive coating to preserve and protect the adhesive. The release liner “r” may be removed prior to adhering the locking base 300 to the body surface “S”. As an alternative, end wall 306 may be coated with adhesive coating 310 prior to placement of locking base 300 on the patient's skin.

Locking segment 304 defines passage or aperture 312 for reception of portal member 204 of portal 200. Locking segment 304 may be a locking collet, clamp or the like. In one embodiment, locking segment 304 is a locking collet having a plurality of collet segments 314 arranged in spaced relation with adjacent collar segments being separated by gaps 316. Collet segments 314 may be selectively displaced in a radial inward direction relative to longitudinal axis “k” to engage portal member 204 in fixed relation therewith. In particular, locking segment or collet 304 is adapted to transition between an initial condition of FIG. 1 permitting passage of portal member 204 through collet passage 312 and a clamped position of FIG. 4 where collet segments 314 selectively engage the outer surface of portal member 204 in secured relation. Locking segment 304 may be biased toward the initial condition of FIG. 1.

Locking base 302 and locking segment 304 may be monolithically formed from a suitable material, or, separately formed and connected through conventional means. Suitable materials include any medical grade material such as stainless steel, titanium, and aluminum; other rigid materials, including polymeric materials such as polycarbonate, polypropylene, and polyethylene; and composites and combinations thereof. Locking segment 304 may include a resilient material whereby after radial inward displacement, collet segments 314 return, under their inherent resiliency, to the initial position of FIG. 1.

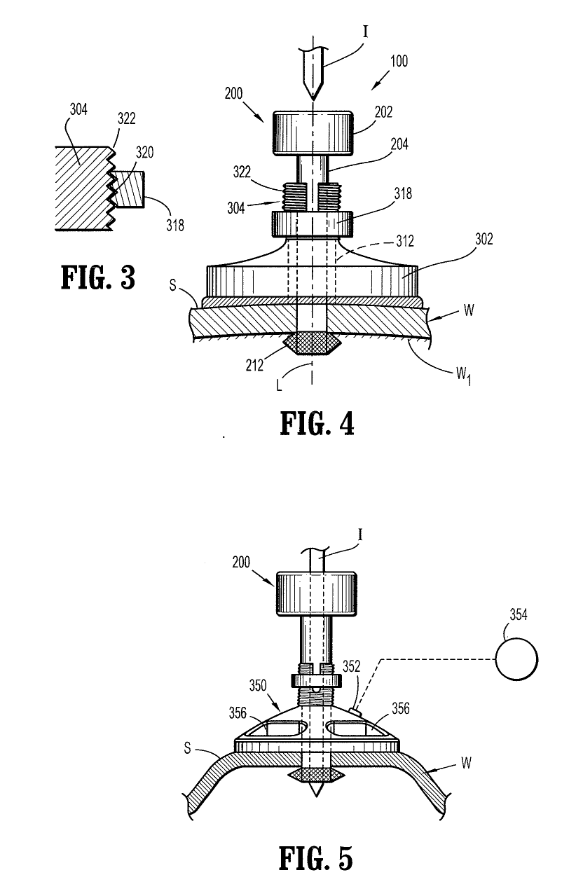

Locking segment or collet 304 is displaceable to assume the clamped condition via a manually manipulative member 318. Manipulative member 318 may be, e.g., a screw, nut, cam member or the like. In one embodiment, manipulative member 318 includes threaded member or nut, which is positionable about locking segment 304. As depicted in the isolated view of FIG. 3, threaded member 318 includes internal threads 320 which cooperatively engage thread segments 322 of locking collet 304. Rotation of threaded member 318 in one direction advances the threaded member 318 toward flange segment 302 and moves collet segments 314 radially inwardly toward the clamped condition. Rotation of threaded member 318 in a second direction cause the threaded member 318 to move away from flange segment 302 permitting collet segments 314 to return toward the initial position thereof.

In another embodiment, manipulative member or threaded member 318 is replaced with a rotatable locking cam defining cam surfaces which may cooperate with corresponding cam surfaces of locking segment 304. In another embodiment, threaded member 318 is replaced with a sleeve devoid of threads. The sleeve may be advanced or retracted along locking segment 304 to move collet segments 314 between the initial and clamped conditions. The sleeve may include an internal taper and may be secured in a position corresponding to the clamped condition of locking segment 304 via a friction fit or the like.

The use of portal apparatus 100 will now be discussed. Flange segment 302 of locking base 300 is positioned against surface S of the body Wall W. The release liner “r” may be removed from contacting surface 308 of locking base 300 to expose adhesive coating 310. Flange segment 302 is applied against the body surface S with the adhesive coating establishing a temporary grip on the surface S. Portal member 204 of portal 200 is introduced within passage of locking segment 304 of locking base 300, and is advanced to a desired longitudinal position with respect to the locking base 300. An obturator may be advanced within portal member 204 to penetrate the body wall W. The obturator is removed. Portal member 204 may be selectively advanced or retracted relative to locking base 300 to position portal 200 at a desired longitudinal position relative to the longitudinal axis “m” of locking base 300 and body wall W. Thereafter, manipulative member 318 is positioned about locking segment 304 and advanced (through e.g., rotation thereof) to traverse the locking segment 304 and cause the locking segment 304 to move toward the clamped condition of FIG. 4. Once achieved, fixation element 212 is activated through, e.g., introduction of fluids within an expandable balloon, or any of the aforementioned means, to engage the internal surface W₁ of body wall W. Thus, with flange segment 302 of locking base 300 engaging surface S of body wall W and fixation element 212 engaging internal surface W₁ of body wall W, portal 200 is secured in fixed relation relative to the body wall W. During the procedure, the longitudinal positioning of portal 200 may be adjusted by release of manipulative member 318 from locking segment 304 thereby permitting portal member 204 to be slid toward or away relative to body wall W and within the locking segment 304. Once the desired relative longitudinal positioning of portal 200 is achieved, manipulative member 318 is moved to cause transitioning of locking segment 304 to the clamped position of FIG. 4. Instruments I are advanced through passage 206 of portal 200 to perform a desired procedure.

Referring now to FIG. 5, another embodiment of locking base 350 of surgical portal system 100. Locking base 350 includes port 352 incorporated into the locking base 350. Port 352 may be used with a source of insufflation fluids 354 to raise, or maintain, the body wall W away from underlying organs thereby providing a cavity in which to operate. For example, a veress needle may be inserted through port 352. In the alternative, port 352 may be connected through a luer fitting or the like with tubing which in turn is in fluid communication with a source of insufflation fluids such as a CO₂ source. One or more apertures 356 may be disposed around the locking base 350 for permitting visualization through locking base 350.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

What is claimed is:

1. A surgical portal apparatus, which comprises:
a portal including an elongated portal member and defining a portal passage for reception of a surgical object;
a locking base including:
a flange segment defining a longitudinal axis and having a longitudinal opening for permitting reciprocal longitudinal passage of the portal member, the flange segment being dimensioned and configured for positioning against body tissue; and
a locking segment mounted to the flange segment, the locking segment dimensioned to selectively engage the portal member and establish a secured relation therewith to selectively secure the portal at a predetermined longitudinal position with respect to the longitudinal axis of the locking base.

2. The surgical portal apparatus according to claim 1, wherein the flange segment of the locking base includes an adhesive coating for adhesive engagement with the body tissue.

3. The surgical portal apparatus according to claim 1, wherein the portal member includes a fixation element, the fixation element dimensioned to engage the body tissue.

4. The surgical portal apparatus according to claim 1, wherein the fixation element includes an expandable element.

5. The surgical portal apparatus according to claim 4, wherein the expandable element is a balloon member.

6. The surgical portal apparatus according to claim 4, wherein the expandable element is an expandable mesh.

7. The surgical portal apparatus according to claim 1, including a manual actuation member mounted adjacent the locking segment, the actuation member movable to bias the locking segment in at least a radial inward direction relative to the longitudinal axis to engage the portal member.

8. The surgical portal apparatus according to claim 7, wherein the locking segment includes a locking collet, the locking collet defining a collet passage for reception of the portal member, the locking collet adapted to move between an initial condition permitting movement of the portal member within the collet passage and a clamped condition securing the portal member relative to the collet passage.

9. The surgical portal apparatus according to claim 8, wherein the locking segment includes collet segments, the collet segments adapted for radial inward movement relative to the longitudinal axis during movement of the actuation member.

10. The surgical portal apparatus according to claim 9, wherein the actuation member includes a threaded member, the threaded member cooperatively engaging corresponding threads of the locking collet.

11. A surgical portal apparatus, wherein:
a portal member defining proximal and distal ends and having a portal passage for reception of a surgical object, the portal member having an expandable fixation element adjacent the distal end and being expandable to engage body tissue;
a locking base including:
flange segment defining a longitudinal axis and having a longitudinal opening for permitting reciprocal longitudinal movement of the portal member, the flange segment dimensioned to engage body tissue opposing the body tissue engaged by the portal member; and
a locking collet defining a collet passage for reception of the portal member, the locking collet adapted to selectively transition between an initial condition permitting passage of the portal member through the collet passage and a clamped condition where the locking collet secures the portal member within the locking base; and
a manual manipulative member mounted to the locking collet, the manipulative member selectively movable to selectively cause transition of the locking collet between the initial condition ad the clamped condition.

12. The surgical portal apparatus according to claim 11 wherein the locking collet is normally biased toward the initial condition.

13. The surgical portal apparatus according to claim 11 wherein the flange segment includes apertures therethrough to permit visualization through the flange segment.

14. The surgical portal apparatus according to claim 11 wherein the locking base includes an insufflation port.
